(19) United States
(12) Patent Application Publication
(10) Pub. No.: US 2023/0399404 A1
(43) Pub. Date: Dec. 14, 2023

(54) SUSTAINED ANTIBODY AND IMMUNOTHERAPEUTIC DELIVERY TO CERVICAL LYMPH NODES

(71) Applicant: The Johns Hopkins University, Baltimore, MD (US)

(72) Inventors: Michael Lim, Stanford, CA (US); John Choi, Stanford, CA (US); Ayush Pant, Baltimore, MD (US); Denis Routkevitch, Baltimore, MD (US); Christopher Jackson, Baltimore, MD (US)

(21) Appl. No.: 18/249,314

(22) PCT Filed: Oct. 19, 2021
(86) PCT No.: PCT/US21/55590

§ 371 Date: Apr. 17, 2023

Related U.S. Application Data
(60) Provisional application No. 63/093,491, filed on Oct. 19, 2020.

(51) Int. Cl. C07K 16/28; A61K 45/06; A61K 47/34; A61K 47/10; A61P 35/00

(52) U.S. Cl. CPC C07K 16/2818; A61K 45/06; A61K 47/34; A61K 47/10; A61P 35/00; A61K 2039/505

(57) ABSTRACT

Methods and compositions for treating a glioblastoma (GBM) by administering a composition comprising a hydrogel and an anti-programmed cell death protein 1 (PD-1) antibody to one or more draining lymph nodes (DLNs) of a subject in need of treatment thereof are disclosed.

### BACKGROUND

Approximately 319,000 people a year are diagnosed with glioblastoma (GBM). Despite the advent of immunotherapy as a promising therapeutic, GBM remains resistant to using checkpoint blockade against programmed cell death protein 1 (PD-1) on T cells. The highly immunosuppressive tumor milieu of GBM prevents rescue of inactivated T cells due to decreased numbers of infiltrating T cells at the tumor site, as well as increased recruitment of myeloid cells. Anti-programmed cell death protein 1 (anti-PD-1) antibody therapy currently involves systemic infusion that is administered every two or three weeks. This systemic administration, however, produces adverse effects on organs, such as the colon, resulting in colitis.

### SUMMARY

In some aspects, the presently disclosed subject matter provides a method for treating a glioblastoma (GBM), the method comprising administering a composition comprising a hydrogel and an anti-programmed cell death protein 1 (PD-1) antibody to one or more draining lymph nodes (DLNs) of a subject in need of treatment thereof. In some aspects, the draining lymph node comprises a cervical lymph node or an inguinal lymph node. In some aspects, the draining lymph node comprises a cervical lymph node. In some aspects, the draining lymph node comprises an inguinal lymph node. In some aspects, the draining lymph node comprises a cervical lymph node and an inguinal lymph node.

In some aspects, the hydrogel comprises an ABA block tripolymer. In certain aspects, the B block of the ABA block tripolymer comprises poly(ethylene glycol) (PEG). In some aspects, the A block of the ABA block tripolymer comprises one or more hydrophobic polymers. In certain aspects, the one or more hydrophobic polymers are selected from poly(ε-caprolactone) (PCL), poly(D,L-lactide-co-glycolic acid) (PLGA), poly (D,L-lactic acid) (PLA), poly(p-phenylene oxide) (PPO), polyhydroxybutyrate (PHB), and combinations thereof. In particular aspects, the hydrogel comprises a poly(ε-caprolactone)-b-poly(ethylene glycol)-b-poly(ε-caprolactone) (PCL:PEG:PCL) triblock polymer or a poly(lactide-co-glycolide)-b-poly(ethylene glycol)-b-poly(lactide-co-glycolide) (PLGA-PEG-PLGA) triblock polymer.

In some aspects, the hydrogel comprises a thermosensitive hydrogel. In some aspects, the hydrogel further comprises one or more pH-sensitive moieties.

In some aspects, the anti-PD-1 antibody is selected from cemiplimab, nivolumab, pembrolizumab, avelumab, atezolizumab, and combinations thereof.

In some aspects, the method further comprises administering the presently disclosed hydrogel composition in combination with one or more therapies for treating a GBM. In some aspects, the one or more therapies for treating a GBM are selected from surgical resection, surgical re-resection, radiation therapy, chemotherapy, vaccine therapy, oncolytic viral therapy, steroid therapy, laser interstitial thermal therapy (LITT), tumor treating fields (TTF) therapy, laser ablation, one or more additional immunotherapies, CSF-1R inhibition, TGF-beta inhibition, IDO-1 inhibition, stromal vascular fraction (SVF) stem cell therapy, stimulator of type-I interferon (IFN) genes) (STING) agonist (cyclic diguanylate monophosphate), and combinations thereof.

In some aspects, the GBM is a O-6-methylguanine-DNA methyltransferase gene (MGMT)-methylated GBM. In other aspects, the GBM has unmethylated/indeterminate MGMT promoter status.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1a, FIG. 1b, FIG. 1c, FIG. 1d, and FIG. 1e demonstrate that hydrogel-mediated release of anti-PD-1 has sustained deposition into local lymph nodes. (FIG. 1a) Day 1 and 9 mouse harvests demonstrating intact presence of hydrogel at the site of the left inguinal lymph node. (FIG. 1B, FIG. 1c) Day 1 and 9 of organ harvests in non-tumor-bearing mice that have been implanted with hydrogels loaded with anti-PD-1 at the site of the left inguinal region (n=3 per arm). (FIG. 1d, FIG. 1e) Day 1 and 9 of organ harvests in non-tumor-bearing mice that have been implanted with hydrogels loaded with anti-PD-1 at the site of the left anterior cervical region (n=3 per arm). (** P<0.01, * P<0.05);

FIG. 2a, FIG. 2b, FIG. 2c, FIG. 2d, FIG. 2e, and FIG. 2f demonstrate that glioma-bearing mice exhibit different distribution of anti-PD-1 in the brain depending on delivery mechanism of anti-PD-1. (FIG. 2a, FIG. 2b) Day 1 and 9 of organ harvests in GL261-bearing mice that have been implanted with hydrogels loaded with anti-PD-1 at the site of the left inguinal region (n=3 per arm). (FIG. 2c, FIG. 2d) Day 1 and 9 of organ harvests in GL261-bearing mice that have been implanted with hydrogels loaded with anti-PD-1 at the site of the left anterior cervical region (n=3 per arm). (FIG. 2e) Comparison between pooled i.p. injection and hydrogel treated mice for anti-PD-1 distribution in the brain (pooled n=6 per arm). (FIG. 2f) Comparison between inguinal lymph node and cervical lymph node treated mice for anti-PD-1 distribution in the brain (n=3 per arm) (** P<0.01, * P<0.05);

FIG. 3a, FIG. 3b, FIG. 3c, and FIG. 3d show flow cytometric analysis of harvested tumor-bearing mouse brain with polymer-based vs systemic vs intracranial delivery of anti-PD-1. Mice were either not treated or given intracranial hydrogel (loaded with 200-μg anti-PD-1), intraperitoneal injection of anti-PD-1 (total dose 600 μg spaced over five days), cervical hydrogel (loaded with 600 μg of anti-PD-1), and inguinal hydrogel (loaded with 600 μg of anti-PD-1). (FIG. 3a) Flow cytometry plots demonstrating % parent populations of CD4+ and CD8+ IFN-γ expression in the brain of mice bearing GBM (n=5 per arm). (FIG. 3b) Summary plots of IFN-γ activity in CD4+ and CD8+ cells based on treatment modality (n=5 per arm). (FIG. 3c) Flow cytometry plots demonstrating % parent populations of CD4+ and CD8+ TNF-α expression in the brain of mice bearing GBM (n=5 per arm). (FIG. 3d) Summary plots of TNF-α activity in CD4+ and CD8+ cells based on treatment modality (n=5 per arm). (*** P<0.001, ** P<0.01, * P<0.05);

FIG. 4a, FIG. 4b, and FIG. 4c demonstrate the survival efficacy of polymer placement involving anti-PD-1. (FIG. 4a) General timeline for dosing schedules for tumor-bearing mice implanted with hydrogel or treated systemically with anti-PD-1. (FIG. 4b) Pictured here are PCL:PEG:PCL hydrogels carrying anti-PD-1 for local delivery to cervical lymph nodes. Without wishing to be bound to any one particular theory, the schematic demonstrates the proposed mechanism of how anti-PD-1 is impacting the T cell compartment. (FIG. 4c) Survival data of mice implanted with hydrogels vs systemic delivery of anti-PD-1 are demonstrated, with mice implanted with hydrogels at the deep cervical lymph nodes demonstrating improved survival efficacy compared to systemic delivery (n=10 per arm) (P=0.0185);

FIG. 5a and FIG. 5b show an in vitro co-culture assay for IFN-γ expression with ELISA. T cells were isolated via FACS microfluidic sorting from mouse cervical and inguinal lymph nodes that were treated with anti-PD-1 monotherapy (intraperitoneal vs polymer placement) and co-cultured with GL261 tumor cell lysate and CD11c+ dendritic cells and plotted for concentration of IFN-γ in supernatant of co-culture wells (n=6 per arm) (* P<0.05);

FIG. 6a, FIG. 6b, and FIG. 6c illustrate the use of gating strategies with markers for determining CD4+ and CD8+ T cell activation to assess the immunogenic potential of the hydrogel platform (FIG. 6a). Fluorescence minus one (FMO) for IFN-γ and TNF-α, immune activation markers that are upregulated in activated immune cells, such as CD4+ and CD8+ T lymphocytes (FIG. 6b), were utilized to indicate proportion of activated cell populations. Representative markers and fluorophores are provided in FIG. 6c;

FIG. 7 shows the immunogenic activation of lymph nodes, including lymphocyte gating strategy, various markers and fluorophores employed, and fluorescence minus one (FMO) flow cytometry measurements; and

FIG. 8 is a survival study using intracranial injections of a STING agonist with or without ReGel®.

### DETAILED DESCRIPTION

The presently disclosed subject matter now will be described more fully hereinafter with reference to the accompanying Examples and Figures, in which some, but not all embodiments of the presently disclosed subject matter are illustrated. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Examples and Figures. Therefore, it is to be understood that the presently disclosed subject matter is not to be limited to the specific embodiments disclosed and that modifications and other embodiments are intended to be included within the scope of the appended claims.

### I. Sustained Antibody and Immunotherapeutic Delivery to Cervical Lymph Nodes

In some embodiments, the presently disclosed subject matter provides a method for treating a glioblastoma (GBM), the method comprising administering a composition comprising a hydrogel and an anti-PD-1 antibody to one or more draining lymph nodes (DLNs) of a subject in need of treatment thereof. In some embodiments, the one or more draining lymph node comprises a tumor-draining lymph node. Generally, a tumor-draining lymph node is a lymph node that is downstream of a tumor site.

In some embodiments, the draining lymph node comprises a cervical lymph node or an inguinal lymph node. In some embodiments, the draining lymph node comprises a cervical lymph node. In some embodiments, the draining lymph node comprises an inguinal lymph node. In some embodiments, the draining lymph node comprises a cervical lymph node and an inguinal lymph node.

Inguinal lymph nodes are lymph nodes located in the groin, whereas cervical lymph nodes are lymph nodes found in the neck. Cervical lymph nodes can be classified in a number of different ways. For example, the American Academy of Otolaryngology system (2002) divides the nodes as follows:

Level I: Submental and submandibular nodes.

Level Ia: Submental—within the triangular boundary of the anterior belly digastric muscles and the hyoid bone.

Level Ib: Submandibular triangle—within the boundaries of the anterior belly of the digastric muscle, the stylohyoid muscle and the body of the mandible.

Level II: Upper jugular nodes (Subdigastric nodes)—around the upper third of the internal jugular vein and adjacent accessory nerve. The upper boundary is the base of the skull and the lower boundary is the inferior border of the hyoid bone. The anterior/medial boundary is the stylohyoid muscle and the posterior/lateral one is the posterior border of the sternocleidomastoid muscle. On imaging the anterior/medial boundary is the vertical plane of the posterior surface of the submandibular gland.

Level IIa: Anterio-medial to the vertical plane of the accessory nerve.

Level IIb: Postero-lateral to this plane.

Level III: Middle jugular nodes—around the middle third of the internal jugular vein, from the inferior border of the hyoid to the inferior border of the cricoid cartilage. Anteromedially they are bounded by the lateral border of the sternohyoid muscle and posterolaterally by the posterior border of the sternocleidomastoid.

Level IV: Lower jugular nodes—around the lower third of the internal jugular vein from the inferior border of the cricoid to the clavicle, anteromedially by the lateral border of the sternohyoid and posterolaterally by the posterior border of the sternocleidomastoid.

Level V: Posterior triangle nodes—around the lower half of the spinal accessory nerve and the transverse cervical artery, and includes the supraclavicular nodes. The upper boundary is the apex formed by the convergence of the sternocleidomastoid and trapezius muscles, and inferiorly by the clavicle. The anteromedial border is the posterior border of the sternocleidomastoid and the posterolateral border is the anterior border of the trapezius.

Level VA: Above the horizontal plane formed by the inferior border of the anterior cricoid arch, including the spinal accessory nodes.

Level VB: Lymph nodes below this plane, including the transverse cervical nodes and supraclavicular nodes (except Virchow's node which is in IV).

Level VI: Anterior compartment nodes—Pretracheal, paratracheal, precricoid (Delphian) and perithyroid nodes, including those on the recurrent laryngeal nerve. The upper border is the hyoid, the lower the suprasternal notch, and the lateral borders the common carotid arteries.

The American Joint Committee on Cancer (AJCC) system differs from that of the American Academy of Otolaryngology system by including Level VII. In the AJCC system, the boundaries are defined as (Superior, Inferior, Antero-medial, Postero-lateral).

Level IA: Symphysis of mandible, Body of hyoid, Anterior belly of contralateral digastric muscle, Anterior belly of ipsilateral digastric muscle.

Level IB: Body of mandible, Posterior belly of digastric muscle, Anterior belly of digastric muscle, Stylohyoid muscle.

Level IIA: Skull base, Horizontal plane defined by the inferior border of the hyoid bone, The stylohyoid muscle, Vertical plane defined by the spinal accessory nerve.

Level IIB: Skull base, Horizontal plane defined by the inferior body of the hyoid bone, Vertical plane defined by the spinal accessory nerve, Lateral border of the sternocleidomastoid muscle.

Level III: Horizontal plane defined by the inferior body of hyoid, Horizontal plane defined by the inferior border of the cricoid cartilage, Lateral border of the sternohyoid muscle, Lateral border of the sternocleidomastoid or sensory branches of cervical plexus.

Level IV: Horizontal plane defined by the inferior border of the cricoid cartilage, Clavicle, Lateral border of the sternohyoid muscle, Lateral border of the sternocleidomastoid or sensory branches of cervical plexus.

Level VA: Apex of the convergence of the sternocleidomastoid and trapezius muscles, Horizontal plane defined by the lower border of the cricoid cartilage, Posterior border of the sternocleidomastoid muscle or sensory branches of cervical plexus, Anterior border of the trapezius muscle.

Level VB: Horizontal plane defined by the lower border of the cricoid cartilage, Clavicle, Posterior border of the sternocleidomastoid muscle, Anterior border of the trapezius muscle.

Level VI: Hyoid bone, Suprasternal notch, Common carotid artery, Common carotid artery.

Level VII: Suprasternal notch, Innominate artery, Sternum, Trachea, esophagus, and prevertebral fascia.

Deep lymph nodes include the submental and submandibular (submaxillary). Anterior cervical lymph nodes (deep) include the prelaryngeal, thyroid, pretracheal, and paratracheal. Deep cervical lymph Nodes include the lateral jugular, anterior jugular, and jugulodigastric. Inferior deep cervical lymph nodes include the juguloomohyoid and the supraclavicular (scalene).

Hydrogel polymers are matrices that demonstrate sustained, localized, and controlled release of bioactive agents. Hydrogel polymers can be chemically and/or physically crosslinked. For example, traditional ReGel® (PLGA-PEG-PLGA) is a physically crosslinked hydrogel that has been previously explored with OncoGel™ (ReGel/paclitaxel). See, for example, Vellimana, et al., 2013.

Hydrogels are useful for drug delivery due to their high biocompatibility and ability to sustain delivery. Examples of hydrogels for drug delivery are provided in the chart immediately herein below (from Larrañeta et al., 2018):

TABLE 1
Representative Examples of Delivery of Therapeutic Agents Using
Hydrogels Containing Hydrophobic Moieties.

| | Therapeutic | | |
|---|---|---|---|
| Hydrogel | Agent | Indication | Administration |
| PMMA/ | Fluorescein | — | Oral |
| PAA-based | | | |
| hydrogel | | | |
| PMA/PEG/ | Fluorescein | — | Oral |
| poly(butyl | | | |
| acrylate)-based | | | |
| hydrogel | | | |
| PCL-PEG-PCL | Docetaxel | Breast cancer | Oral |
| Hydrogels | Doxorubicin | Colon cancer | Oral |
| containing | | | |
| PMMA | | | |
| nanoparticles | | | |
| Silicone-based | Atropine | — | Ocular |
| hydrogel | | | (contact |
| | | | lenses) |
| HEMA-based | Lidocaine | — | Ocular (contact |
| hydrogels | | | lenses) |
| containing | | | |
| microemulsions | | | |
| HEMA-based | Cyclosporine A | — | Ocular (contact |
| hydrogels | | | lenses) |
| containing | | | |
| microemulsions | | | |
| HEMA-based | Loteprednol | — | Ocular (contact |
| hydrogels | etabonate | | lenses) |
| containing | | | |
| microemulsions | | | |
| HEMA-based | Diclofenac | Ocular | Ocular (contact |
| hydrogels | | inflammatory | lenses) |
| containing | | disorders | |
| CDs | | | |
| PEG/silica | Dexamethasone | — | Ocular |
| hydrogel | | | (injectable |
| | | | implant) |
| Poly(trimethylene | Mitomycin C | — | Ocular |
| carbonate)/ | | | (injectable |
| Pluronic | | | implant) |
| F127 hydrogel | | | |
| Cellulose/ | Isoliquiritigenin | Anti-microbial | Topical |
| hyaluronic | | therapy | |
| acid-based | | | |
| hydrogel | | | |
| Carbomer | Penciclovir | Anti-viral | Topical |
| 940-based | | therapy | |
| gel containing a | | | |
| microemulsion | | | |
| PEG-PCL-based | Curcumin | Wound | Topical |
| hydrogel | | healing | |
| Carbomer | Astragaloside IV | Wound | Topical |
| 940-based | | healing | |
| gel containing | | | |
| solid lipid | | | |
| nanoparticles | | | |
| Carbopol 934, | Nitrendipine | Hypertension | Transdermal |
| xanthan gum, | | | |
| hydroxy propyl | | | |
| cellulose (HPC) | | | |
| and | | | |
| chitosan-based | | | |
| hydrogels | | | |
| containing | | | |
| nanostructured | | | |
| lipid | | | |
| carriers | | | |
| Pluronic-based | Nitrendipine | Solid tumors | Transdermal |
| hydrogel | | | |

Hydrogels have been traditionally used for delivery of hydrophilic drugs, but PCL-PEG-PCL tri-block polymers have had success with hydrophobic compounds by copolymerizing lactide with the more hydrophilic glycolide to create Poly(lactic co glycolic acid) (PLGA). ReGel®, a triblock copolymer arranged as PLGA-PEG-PLGA, is a free flowing water soluble solution at low temperatures (about 2° C. to about 15° C.) that transitions to a gel at body temperature (about 37° C.). Hydrogels having the alternate arrangement PEG-PLGA-PEG have been found to have similar properties to ReGel®, e.g., a sol-gel transition temperature of about 37° C.

PCL-PEG-PCL tri-block hydrogel is a thermosensitive hydrogel that increases the solubility of hydrophobic compounds because it possesses a hydrophobic core while still being able to deliver hydrophilic compounds.

Thermosensitive hydrogels respond to changes in temperature and usually undergo a sol-gel phase transition when the temperature changes from room temperature to physiological temperature. This property makes them particularly useful for drug delivery, as temperature is generally an easy stimulus to control.

Thermosensitive hydrogels are usually triblock polymers made up from poly(ethylene glycol) (PEG) linked to hydrophobic polymer blocks. The triblock is composed of A blocks and B blocks organized as ABA or BAB. Compositions having PEG as the A block are well-established for use in hydrogel formulation as PEG possesses high water solubility, biocompatibility, and low immunogenicity. The B blocks increase the hydrophobicity and drug loading capacity of hydrophobic drugs by micellization. Representative block copolymers used for the preparation of physical hydrogels are shown immediately herein below, and including poly(p-phenylene oxide) (PPO), poly (D,L-lactic acid) (PLA), poly(D,L-lactide-co-glycolic acid) (PLGA), poly(ε-caprolactone) (PCL), and polyhydroxybutyrate (PUB):

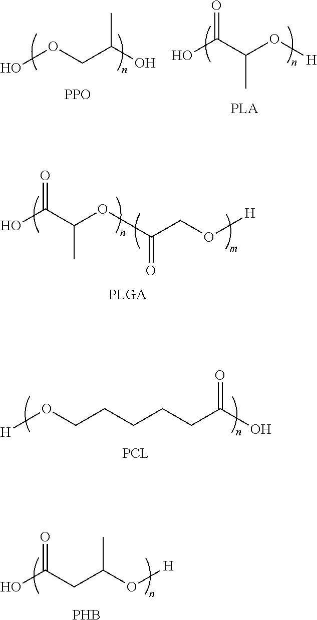

In some embodiments, the thermogelling hydrogel comprises triblock copolymers of PEG and poly(lactic acid) (PLA) in the following orientation PLA-PEG-PLA. These hydrogels have been significantly studied and are used widely for drug delivery applications due to their biodegradability and biocompatibility properties and their valuable ability to self-assemble in aqueous media to form polymeric micelles with a core-shell structure.

In certain embodiments, ultraviolet irradiation can be used to photo-crosslink PLA-PEG-PLA hydrogels with acrylated end groups. In other embodiments, the PLA-PEG-PLA hydrogels are not photo-crosslinked but instead can be synthesized by the nanoprecipitation method, with the nanogel being formed by thermal crosslinking.

Administration of thermosensitive hydrogels using a syringe/needle can be problematic because of their gelation transition temperature of about 37° C. Under such circumstances, a patient's body temperature can cause the rapid gelation of the hydrogel, thereby blocking the needle. To overcome this issue, pH-sensitive moieties can be added to existing thermosensitive copolymers, so for gelation to occur a second condition must be met, i.e., pH.

One approach to generate gels that are thermo- and pH-sensitive is though the addition of sulfamethazine oligomers (OSM) endcaps to already thermosensitive copolymers. An example of this is the addition of OSM endcaps to the thermosensitive parent triblock poly(ε-caprolactone-co-lactide)-b-poly(ethylene glycol)-b-poly(ε-caprolactone-co-lactide) (PCLA-PEG-PCLA) to form OSM-PCLA-PEG-PCLA-OSM. When the formulation is at room temperature and at pH 8.0 it is in the sol-state, to transition to the gel-state, two conditions must be met, the temperature must rise to 37° C. and the pH must drop to pH 7.4. This means the needle will not become blocked during administration as only increasing the temperature will not cause the change to the gel form on its own, the pH must also drop to 7.4 inside the needle, which does not occur. Thus, this formulation may be employed as injectable carriers for hydrophobic drugs.

The triblock copolymers consisting of PEG as the A Block and poly(acrylic acid) PAA as the B block arranged as BAB has been shown to have a thermo- and pH-sensitive nature. At relatively low pHs, e.g., pH 3.0, the PAA block is hydrophilic but, at higher pHs e.g., pH 7.4, the PAA block becomes hydrophobic. Furthermore, as the temperature increases, the pKa of the PAA-PEG-PAA polymer decreases, indicating that an increase in temperature, increases the hydrophobicity. The PAA-PEG-PAA polymer was found to undergo a sol-to-gel-to-condensed gel transition at pH 7.4 and at 37° C., with the condensed gel having a high viscosity of 43.6 kPa·s.

The addition of poly(β-amino ester) (PAE) endcaps to a PCL-PEG-PCL triblock imparts a pH-sensitive nature, in addition to the thermosensitive properties already held by the parent triblock. The parent block alone only transitions in response to temperature (gel region of about 34° C. to about 54° C.), but the addition of PAE endcaps to form a pentablock (PAE-PCL-PEG-PCL-PAE), leads to the hydrogel being sensitive to both pH and temperature changes. This formulation was found to undergo sol-gel phase transition above pH 6.0 in response to increasing both temperature and pH.

In some embodiments, the hydrogel comprises an ABA block tripolymer. In some embodiments, the B block of the ABA block tripolymer comprises poly(ethylene glycol) (PEG). In some embodiments, the A block of the ABA block tripolymer comprises one or more hydrophobic polymers. In certain embodiments, the one or more hydrophobic polymers are selected from poly(ε-caprolactone) (PCL), poly(D,L-lactide-co-glycolic acid) (PLGA), poly (D,L-lactic acid) (PLA), poly(p-phenylene oxide) (PPO), polyhydroxybutyrate (PHB), and combinations thereof. In particular embodiments, the hydrogel comprises a poly(ε-caprolactone)-b-poly(ethylene glycol)-b-poly(ε-caprolactone) (PCL:PEG:PCL) triblock polymer or a poly(lactide-co-glycolide)-b-poly(ethylene glycol)-b-poly(lactide-co-glycolide) (PLGA-PEG-PLGA) triblock polymer, structures of which are provided immediately herein below:

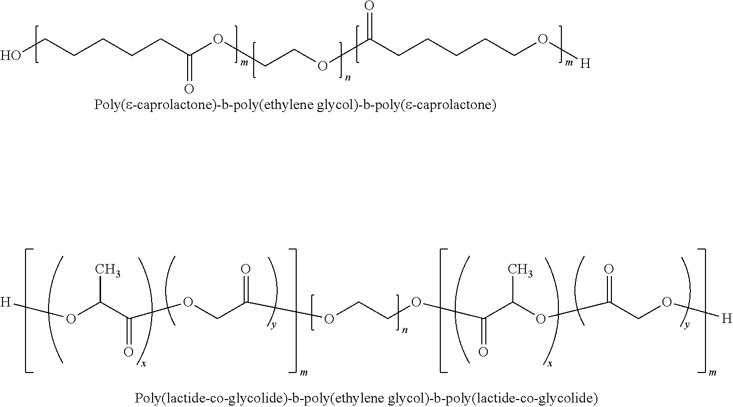

In some embodiments, the hydrogel comprises a thermosensitive hydrogel. In some embodiments, the hydrogel further comprises one or more pH-sensitive moieties.

Representative anti-PD-1 monoclonal antibodies include, but are not limited to, cemiplimab (Libtayo®), nivolumab (Opdivo®), pembrolizumab (Keytruda®), avelumab (Bavencio®), durvalumab (Imfinzi®), and atezolizumab (Tecentriq®).

Generally, anti-PD-1 antibodies are checkpoint inhibitors. A checkpoint inhibitor blocks proteins called checkpoints that are made by some types of immune system cells, such as T cells, and some cancer cells. These checkpoints help keep immune responses from being too strong and sometimes can keep T cells from killing cancer cells. When these checkpoints are blocked, the ability of T cells to kill cancer cells is enhanced. Examples of checkpoint proteins found on T cells or cancer cells include PD-1/PD-L1 and CTLA-4/B7-1/B7-2.

In some embodiments, the method further comprising administering the presently disclosed hydrogel composition in combination with one or more therapies for treating a GBM.

The term “combination” is used in its broadest sense and means that a subject is administered at least two agents, more particularly a hydrogel composition comprising an anti-PD-1 antibody and at least one additional therapeutic agent or in combination with one or more therapeutic method for treating a GBM. More particularly, the term “in combination” refers to the concomitant administration of two (or more) active agents or therapeutic methods for the treatment of a, e.g., single disease state. As used herein, the active agents or therapeutic methods may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the hydrogel composition described herein can be administered alone or in combination with adjuvants that enhance stability of the hydrogel composition, alone or in combination with one or more agents for treating pain, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a hydrogel composition and at least one additional therapeutic agent and/or therapeutic method can be varied so long as the beneficial effects of the combination of these agents and/or methods are achieved. Accordingly, the phrase “in combination with” refers to the administration of a hydrogel composition and at least one additional therapeutic agent and/or therapeutic method either simultaneously, sequentially, or a combination thereof. Therefore, a subject administered a combination of a hydrogel composition and at least one additional therapeutic agent and/or therapeutic method can receive hydrogel composition and at least one additional therapeutic agent and/or therapeutic method at the same time (i.e., simultaneously) or at different times (i.e., sequentially, in either order, on the same day or on different days), so long as the effect of the combination of both agents is achieved in the subject.

When administered sequentially, the agents and/or methods can be administered within 1, 5, 10, 30, 60, 120, 180, 240 minutes or longer of one another. In other embodiments, agents administered sequentially, can be administered within 1, 5, 10, 15, 20 or more days of one another. Where the hydrogel composition and at least one additional therapeutic agent and/or method are administered simultaneously, they can be administered to the subject as separate pharmaceutical compositions, each comprising either a hydrogel composition or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents and/or therapeutic methods can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a hydrogel composition and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In some embodiments, the one or more therapies for treating a GBM are selected from surgical resection, surgical re-resection, radiation therapy, chemotherapy, vaccine therapy (e.g., HSPPC-96, dendritic cell vaccines (e.g., pp65 DC), and CDVAX-L), oncolytic viral therapy (e.g., DNX-2401), steroid therapy, laser interstitial thermal therapy (LITT), tumor treating fields (TTF) therapy, laser ablation, one or more additional immunotherapies, CSF-1R inhibition (e.g., BLZ945, FPA008), TGF-beta inhibition (e.g., galunisertib), IDO-1 inhibition (e.g., indoximod), stromal vascular fraction (SVF) stem cell therapy, stimulator of type-I interferon (IFN) genes) (STING) agonist (e.g., cyclic diguanylate monophosphate), and combinations thereof.

In some embodiments, the radiation therapy comprises one or more of X-ray radiation, gamma ray radiation, and proton beam radiation therapy. In certain embodiments, the radiation therapy comprises one or more of intensity-modulated radiation therapy (IMRT), tomotherapy, stereotactic radiosurgery, hypofractionated stereotactic radiosurgery, stereotactic radiosurgery with valproic acid, and pencil beam proton therapy.

In some embodiments, the chemotherapy comprises an alkylating agent. In certain embodiments, the alkylating agent is selected from temozolomide (Temodar®), lomustine, carmustine, procarbazine, vincristine, and combinations thereof.

In some embodiments, the steroid therapy comprises or more of dexamethasone, prednisone, and combinations thereof.

In some embodiments, the one or more additional immunotherapies are selected from one or more additional checkpoint inhibitors, one or more vascular endothelial growth factor (VEGF) antagonists, one or more cytotoxic T-lymphocyte-associated protein 4 (CTLA-4) inhibitors, one or more vascular endothelial growth factor receptor (VEGFR) inhibitors, CAR-T cell therapy, and combinations thereof.

In some embodiments, the one or more checkpoint inhibitors is selected from anti-PD-L1 (e.g., durvalumab), anti-LAG-3 (e.g., BMS 986016), anti-CD137 (e.g., urelumab), anti-CD-27 (e.g, varlilumab), intratumoral IDO1 inhibitor (e.g., INT230-6), IDO1 inhibitor (e.g, epacadostat), and combinations thereof.

In some embodiments, the one or more VEGF antagonists comprises bevacizumab (Avastin®).

In some embodiments, the one or more CTLA-4 inhibitors are selected from ipilimumab (Yervoy®), tremelimumab, and combinations thereof.

In some embodiments, the one or more vascular endothelial growth factor receptor (VEGFR) inhibitors comprises cediranib.

In some embodiments, the GBM is selected from O-6-methylguanine-DNA methyltransferase gene (MGMT)-methylated GBM and GBM having unmethylated/indeterminate MGMT promoter status.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1 from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Sustained Localized Delivery of Immunotherapy to Lymph Nodes Reverses Immunosuppression and Increases Long-Term Survival in Murine Glioblastoma

### 1.1 Overview

Despite the advent of immunotherapy as a promising therapeutic, glioblastoma (GBM) remains resistant to using checkpoint blockade due to its highly immunosuppressive tumor milieu. Moreover, current anti-PD-1 treatment requires multiple infusions with adverse systemic effects. Therefore, the presently disclosed subject matter, in some embodiments, employs a PCL:PEG:PCL polymer gel loaded with anti-PD-1 and implanted at the site of lymph nodes in an attempt to maximize targeting of inactivated T cells, as well as mitigate unnecessary systemic exposure.

Mice orthotopically implanted with GL261 glioma cells were injected with hydrogels loaded with anti-PD-1 in one of the following locations: cervical lymph nodes, inguinal lymph nodes, and the tumor site. Mice treated systemically with anti-PD-1 were used as comparative controls. Kaplan-Meier curves were generated for all arms, with ex vivo flow cytometric staining for L/D, CD45, CD3, CD4, CD8, TNF-α and IFN-γ and co-culture ELISpots were done for immune cell activation assays.

Mice implanted with PCL:PEG:PCL hydrogels carrying anti-PD-1 at the site of their lymph nodes showed significantly improved survival outcomes compared to mice systemically treated with anti-PD-1 (P=0.0185). Flow cytometric analysis of brain tissue and co-culture of lymph node T cells from mice implanted with gels demonstrated increased levels of IFN-γ and TNF-α compared to mice treated with systemic anti-PD-1, indicating greater reversal of immunosuppression compared to systemic treatment.

The presently disclosed data demonstrate proof of principle for using localized therapy that targets lymph nodes for GBM. This approach provides an alternative treatment paradigm for developing new sustained local treatments with immunotherapy that are able to eliminate the need for multiple systemic infusions and their off-target effects.

### 1.2 Background

Immunotherapy has revolutionized the treatment of cancers across multiple subtypes. Goldberg et al., 2007; Tsushima et al., 2007; Topalian et al., 2020; Blackburn et al., 2010. The use of immune checkpoint blockade (ICB), most notably directed at Programmed Cell Death Protein-1 receptor (PD-1) and cytotoxic T-lymphocyte-associated protein 4 (CTLA-4), rejuvenate anti-tumor responses in inactivated T cells and demonstrate great promise in treating tumors, such as melanoma and non-small cell lung cancer. Khan, 2015; Reck et al., 2016; Larkin et al., 2019.

Despite the success of ICB therapy in these cancers, however, glioblastoma (GBM) remains resistant to current immunotherapeutic strategies due to its immunosuppressive milieu preventing rescue of inactivated T cells and myeloid cells. Jackson et al., 2019. Additionally, current anti-PD-1 therapy is dose-limited by immune-related adverse events (irAEs). Despite the synergistic therapeutic benefit of targeting multiple checkpoint molecules for GBM, Woroniecka et al., 2018, irAEs, such as nephritis and pruritus, are especially pronounced in combination immunotherapy, with several other documented sequelae, such as pneumonitis, colitis, vitiligo, and hypophysitis, seen as consequences of checkpoint blockade. Almutairi et al., 2020. Such irAEs are particularly an issue for GBM because anti-PD-1 monotherapy alone is unlikely to offer sufficient therapeutic benefit. Kim et al., 2017.

Enhancing localized, tumor-targeting immunotherapy would be an important leap forward in cancer immunotherapy that would reduce off-target toxicities while increasing drug efficacy to allow for higher doses of ICB or combination therapy. Many modalities have been tested to achieve this goal, from using irradiated tumor cells to secrete monoclonal antibodies against CTLA-4 at the immunization site to intratumoral injection of adoptively transferred immune cells, such as dendritic cells. Rotman et al., 2019.

### 1.3 Scope

The presently disclosed subject matter examines the potential of targeting lymph nodes, sites of antigen presentation and cytotoxic immune cell activation, using hydrogels that can offer sustained, localized delivery of antibodies, such as anti-PD-1. Since the targets are inactivated T cells, the tumor-draining lymph nodes act as an optimal alternative to targeting the tumor itself; as the site of T cell activation with robust trafficking of professional antigen presenting cells, there is a high likelihood of interacting with immune cells that have the potential for re-activation and expansion of anti-tumor phenotypes, moreover, as a hub of multiple immune cells including myeloid cells, lymph node exposure to immunotherapeutic agents, such as anti-PD-1, into the lymph nodes, also would target additional cell types that might benefit from checkpoint blockade. Roberts et al., 2016; Salmon et al., 2016; Fransen et al., 2018; Strauss et al., 2020. The presently disclosed strategy of using sustained localized delivery of anti-PD-1 to lymph nodes was explored in a murine preclinical model, providing survival data and immunophenotype analysis of its benefits over systemic therapy.

### 1.4 Materials and Methods

### 1.4.1 Murine Glioma Model and Cell Lines

Female 6-8 week-old C57BL/6 J wild-type mice were maintained at the Johns Hopkins University Animal Facility per the Institutional Animal Care and Use Committee (IACUC) protocol. For all in vivo experiments, mice were anesthetized with Ketathesia (100 mg/kg)/xylazine (10 mg/kg) via intra-peritoneal (i.p.) injection and had topical eye gel for lubrication while anesthetized. Mice were placed on a heating pad and observed until fully recovered. GL261-Luc2 cells grown in DMEM (Life Technologies)+10% FBS (Sigma-Aldrich)+1% penicillin-streptomycin (Life Technologies) were used for orthotopic murine glioma models, as described in previous studies. Zeng et al., 2013. 1.3e5 GL261-Luc2 cells in a volume of 2 μL were stereotactically injected 2-mm lateral to the sagittal suture, 2-mm posterior to the coronal suture, and 3-mm deep to the cortical surface in the area of the left striatum. After implantation of tumor cells, mice were assessed for tumor growth on post-implantation day 7 using bioluminescent IVIS® imaging (PerkinElmer).

Mice with sufficient tumor burden at day 7 were then randomly separated into control (non-treated) and treatment arms. The presence of tumor was then monitored by IVIS® imaging on post-implantation days 14, 21, 28, and 40. Survival experiments were repeated in triplicate with 8-10 mice in each control or treatment arm. Animals were euthanized according to humane endpoints, including CNS disturbances, hunched posture, lethargy, weight loss, and inability to ambulate per our IACUC protocol.

Flank tumor models involved female 6-8 week-old C57BL/6 J wild-type mice that were subcutaneously injected in the left hind limb with 10⁶ GL262 cells in 100 μL of mixed PBS and Matrigel (BD Biosciences) in a 1:1 ratio. Mice were treated either intra-peritoneally with anti-PD-1 on days 10, 12, and 14, or implanted with hydrogel loaded-anti-PD-1 (see immediately herein below in section 1.4.2 Therapeutic antibodies) on day 10 at region of the left inguinal lymph node. Control mice were not treated with anti-PD-1. Tumor growth was measured every 2 days using calipers and tumor volumes were calculated in three dimensions using the formula: 4/3πr. Topalian et al., 2020.

### 1.4.2 Therapeutic Antibodies

G4 hybridomas were cultured and used to develop hamster monoclonal antibodies (mAbs) against murine PD-1, as described in previous studies. Hirano et al., 2005. Therapeutic murine antibodies were subsequently stored in 1-mg/mL and 3-mg/mL aliquots at −80° C. To concentrate anti-PD-1 for hydrogel mixtures, 15-mL AMICON ultrafiltration tubes were used to concentrate anti-PD-1 into 100-μg/ilL solutions (MilliporeSigma), with concentrations determined by Nanodrop (Wilmington, DE, USA).

Individual treatment dose was 200 μg per dose on post-implantation days 10, 12, and 14 for anti-PD-1 i.p. injections and 600 μg total for PCL:PEG:PCL hydrogel implantations. Mice implanted with hydrogels loaded with anti-PD-1 were given 50 μL of hydrogel carrying 600-μg anti-PD-1. Hydrogels were implanted <3 mm superficial to the inguinal and deep cervical lymph nodes in the left groin and neck, respectively.

### 1.4.3 Reconstitution of Hydrogel

The PCL:PEG:PCL hydrogel was generously provided to the laboratory of Dr.

Michael Lim by BTG plc. The aqueous solution of the hydrogel was stored at −20° C. and left at room temperature (25° C.) overnight. Aliquots were heated in a 60° C. water bath for 20 minutes with intermittent vigorous shaking (every 2 minutes). Afterward, the bottle was left to stand at 25° C. for 3 hours and then transferred to 4° C. for 2 hours. At this point, the polymer was a clear liquid that was mixed with the appropriate concentration of PD-1 antibody to create 50-4, aliquots of the gel. All aliquots were stored on ice and filtered through a 0.22-μm syringe filter.

### 1.4.4. Immune Cell Harvest and Isolation

Mice were deeply anesthetized or euthanized before harvesting lymph nodes or brains for immunological assays per an IACUC protocol. Red blood cells in brain and lymph node samples were lysed using ACK lysis buffer (ThermoFisher) and resuspended in phosphate buffered saline (PBS) buffer for further cytometric staining. Brains were removed, tissue was mechanically dissociated through a 70-μm filter, and homogenates were centrifuged in a 30%/70% Percoll® (Sigma-Aldrich) gradient at 2200 rpm for minutes without brakes to separate out brain myeloid cells and lymphocytes from tumor cells and myelin. Brain immune cells were extracted at the 30%/70% interface and resuspended in PBS buffer (Sigma-Aldrich) for further cytometric analysis. Lymph nodes were mechanically dissociated through a 70-μm filter, centrifuged at 300 g, and washed in PBS buffer for further cytometric staining and analysis.

### 1.4.5 Flow Cytometric Analysis of Murine Immune Cells

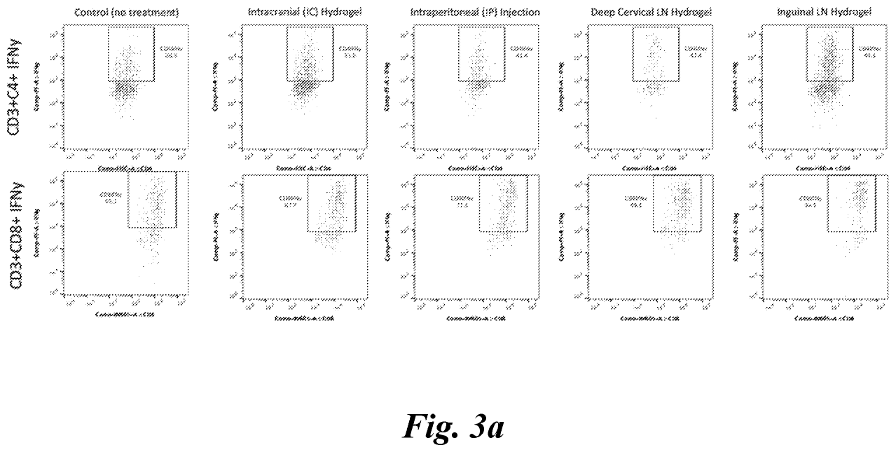

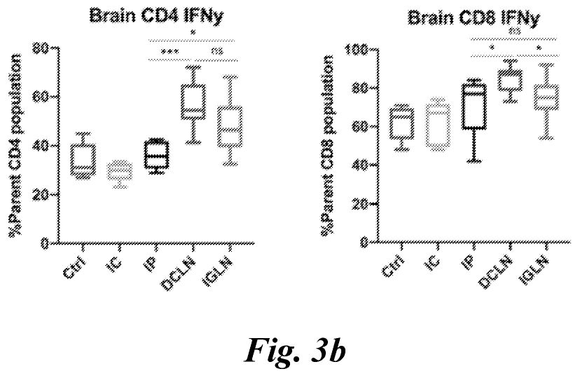

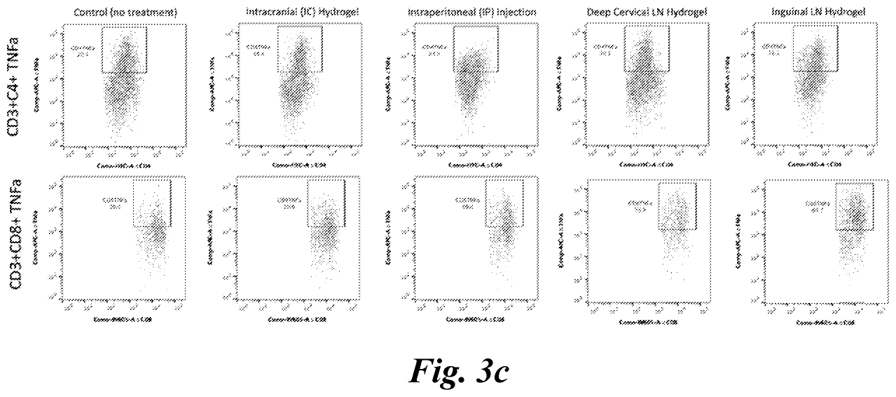

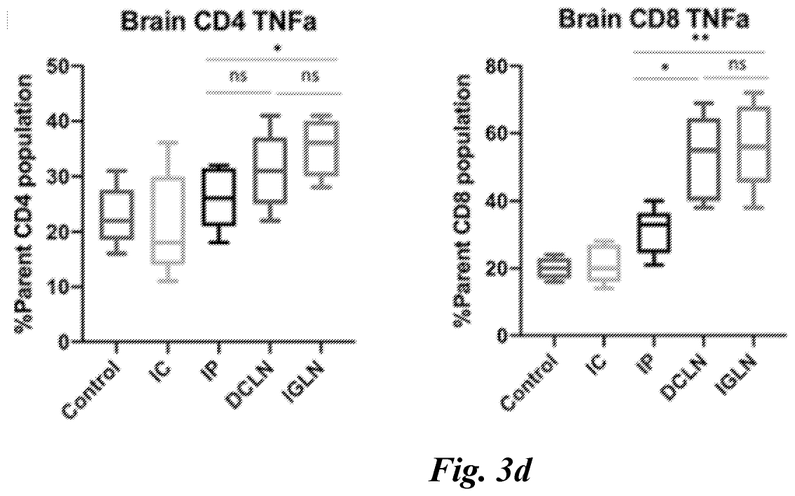

Mouse immune cells were stained for Live/Dead (L/D) (Invitrogen), CD45, CD3, CD4, CD8, IFN-γ, and TNF-α (FIG. 3). To stain for the intracellular marker IFN-γ and TNF-α, samples were fixed in 1:3 fixation/permeabilization concentrate: diluent mixture (eBioscience) for 30 minutes and subsequently stained in permeabilization buffer (eBioscience). Fluorescence minus one (FMO) was used to control for data spread due to multiple fluorochromes and nonbinary expression of markers, such as IFN-γ and TNF-α. Flow data were acquired using a FACSCelesta flow cytometer (BD) and analyzed using FlowJo (BD). Nonviable cells and doublets were excluded by forward versus side scatter gating, forward scatter height versus forward scatter area gating, and L/D staining.

### 1.4.6 Radiolabeling and Radiotracking Harvest Experiments

Using previously described methods, the anti-PD-1 antibody was conjugated to N-[2-amino-3-(p-isothiocyanatophenyl)propyl]-trans-cyclohexane-1,2-diamine-N,N′,N′,N″,N″-pentaacetic acid (SCN-CHX-A″-DTPA) with Indium¹¹¹ added to an acid washed solution containing antibody. Brechbiel and Gansow, 1992; Chongsathidkiet et al., 2018. The mixture was set at 25° C. for 1 h and then transferred to an Amicon Ultrafiltration device with the protein concentration determined by Nanodrop (Wilmington, DE, USA). A 200-μg aliquot of the conjugated anti-PD-1 antibody was mixed with the hydrogel in a 1:3 ratio and implanted into the left inguinal or anterior cervical regions of mice. On days 1 and 9, mice were harvested for their blood, liver, spleen, kidney, bone, deep cervical lymph nodes, inguinal lymph nodes, and brain and measured by weight and gamma well counter using a 400 keV to 480 keV energy window (PerkinElmer 2470 WIZARD2® Automatic Gamma Counter, MA, USA). The percent-injected activity per gram (% IA/g) was calculated by comparison to a weighted, diluted standard.

### 1.4.7 Co-Culture and ELISpot

For IFN-γ assays, 5e3 CD45.2+ CD3+ T cells were sorted from either inguinal or deep cervical lymph nodes in tumor-bearing mice on post-implantation day 14 that received no treatment, treatment with i.p. injected anti-PD-1, treatment with hydrogels at the inguinal lymph node, or treatment with hydrogels adjacent to the deep cervical lymph nodes. These T cells were co-cultured with 100-μg/mL GL261-Luc2 tumor cell lysate and 25e3 dendritic cells isolated from CD45.1 mouse spleen (isolated with a pan-dendritic cell isolation kit) (Miltenyi Biotec) in a 96-well round bottomed plate with T cell media (RPMI 1640+10% FBS+1% NEAA+1% 2-Mercaptoethanol+1% Penicillin/Streptomycin). Co-cultured cells were incubated at 37° C. for 48 hours. Supernatant was collected for subsequent ELISA for IFN-γ and run on a plate reader (Thermo Fisher).

### 1.4.8 Statistical Analysis

All replicates were biological replicates. Survival was analyzed via Kaplan—Meier method and compared by log-rank (Mantel-Cox) test. Calculated variables were treated as continuous variables under the assumption that data follow Student T-distribution. Mouse experimental data were analyzed using a two-tailed Student's T-test for experiments containing two groups. Comparisons between groups were presented as mean±SEM. All data were analyzed using GraphPad Prism 8 and values of P<0.05 were considered statistically significant.

### 1.5 Results

### 1.5.1 Hydrogel-Mediated Release of Anti-PD-1 Results in Sustained Delivery of Antibody to Nearby Lymph Nodes and to Glioblastoma

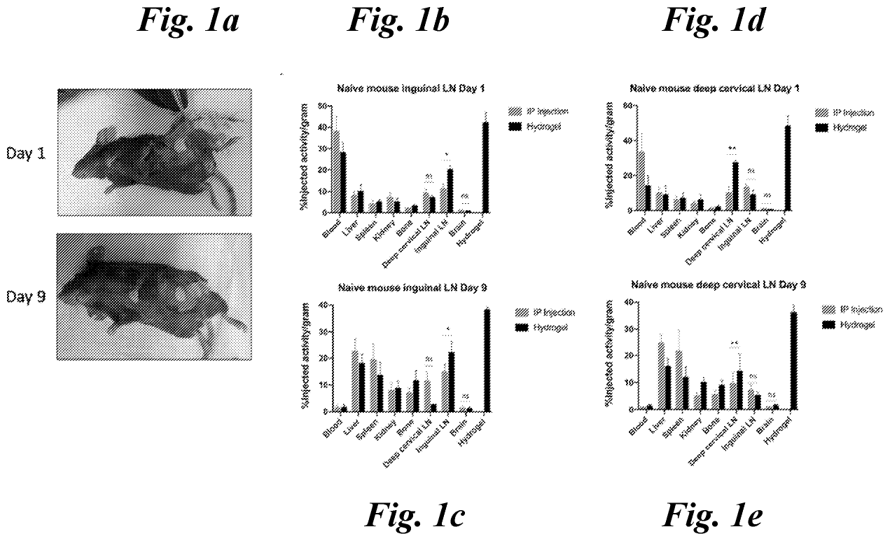

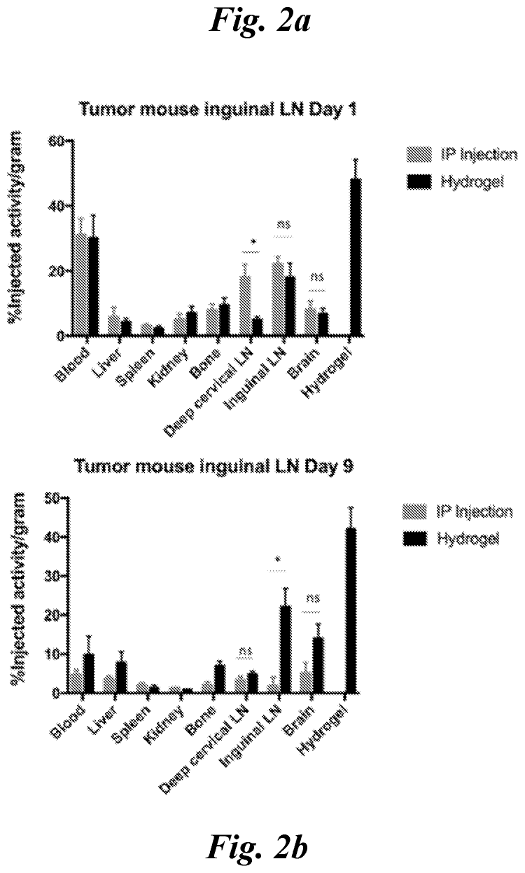

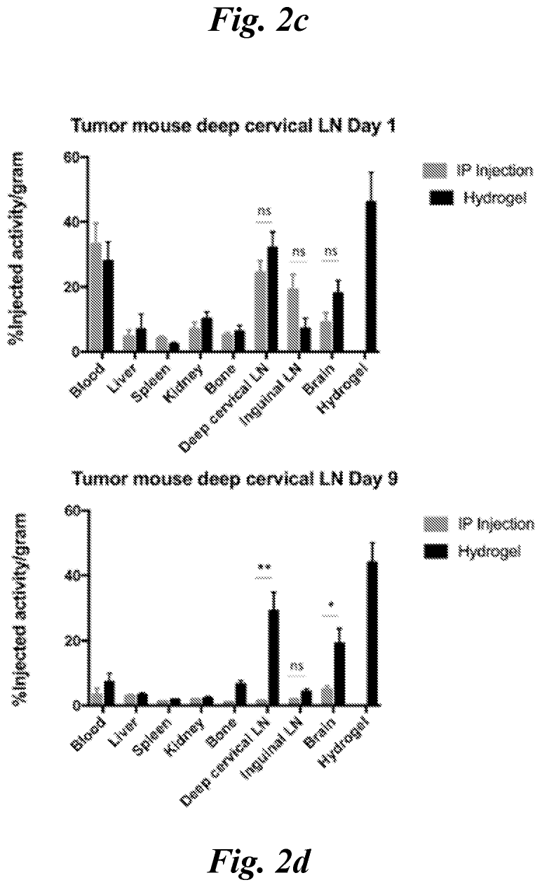

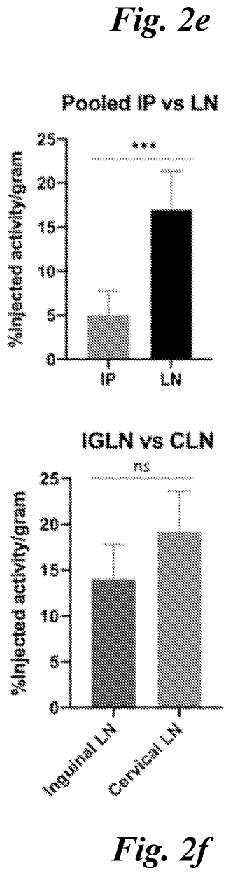

To evaluate whether the PCL:PEG:PCL hydrogel could facilitate sustained delivery of anti-PD-1 antibody to lymph nodes in vivo, mice were injected with hydrogels loaded with ¹¹¹In-DTPA-anti-PD-1 in their inguinal and anterior cervical regions in both the absence (FIG. 1) and presence (FIG. 2) of tumor. The physical integrity of the hydrogel remained intact for the duration of the 9 days between initial and final organ harvests for detecting anti-PD-1 (FIG. 1a).

Non-tumor-bearing mice injected with loaded hydrogels in the inguinal region demonstrated localization of anti-PD-1 to the inguinal lymph nodes when compared to i.p. injection on both day 1 (P=0.0369) and day 9 (P=0.0316) post-injection (FIG. 1B, FIG. 1c). There was no significant difference in percent-injected activity per gram (% IA/g) in the cervical lymph nodes for administration of anti-PD-1 through i.p. or inguinal hydrogel routes (FIG. 1B, FIG. 1c). Within the non-tumor-bearing mice that were injected at the anterior cervical site, % IA/g was significantly higher in the deep cervical lymph nodes for mice that had cervical hydrogel placement compared to i.p. delivery on both day 1 (P=0.0095) and day 9 (P=0.0021). Of note, there were no significant differences in the % IA/g of anti-PD-1 in the brain from i.p. vs hydrogel-based delivery for any of the non-tumor-bearing groups (FIG. 1B-FIG. 1d).

Similarly, mice bearing intracranial glioma demonstrated sustained delivery of anti-PD-1 to their local lymph nodes (FIG. 2). Interestingly, glioma-bearing mice implanted with hydrogel in the inguinal region on day 1 had lower concentrations of anti-PD-1 in their deep cervical lymph nodes (FIG. 2a) than those mice given i.p. anti-PD-1. Otherwise, there were no significant differences between anti-PD-1 distribution from i.p. and hydrogel delivery routes on day 1 (FIG. 2a-FIG. 2c). On day 9, sustained delivery to nearby lymph nodes was noted for tumor-bearing mice with hydrogel injected; mice with hydrogel at their inguinal region had significantly greater % IA/g of anti-PD-1 in their inguinal lymph nodes, while the deep cervical lymph nodes showed comparable levels of % IA/g between i.p. and hydrogel arms (FIG. 2b). In a similar manner, tumor-bearing mice with injection of hydrogel in the anterior cervical region demonstrated increased representation of anti-PD-1 in the deep cervical lymph nodes, but minimal activity in the inguinal lymph nodes (FIG. 2d). Of note, tumor-bearing mice with hydrogel in the cervical region had a significantly different level of anti-PD-1 in the brain on day 9 when compared to mice treated with anti-PD-1 (FIG. 2d).

Although the increase in anti-PD-1 levels in the brain of inguinal hydrogel-treated mice compared to i.p. treated mice was not statistically significant in FIG. 2b, pooling i.p. treated mice with brain tumors from FIG. 2b and FIG. 2d (both harvested day 9 after i.p. injection) shows a statistically significant difference in anti-PD-1 levels in brain between i.p. injection and lymph node implantation (P=0.0002, FIG. 2e). In addition, the % IA/g of anti-PD-1 in the brain of inguinal hydrogel-injected mice was not significantly different from that of cervical hydrogel-injected mice, suggesting that sustained anti-PD-1 delivered locally to lymph nodes eventually accumulates in brain tumors regardless of the target lymph node (P=0.1641, FIG. 2f).

Notably, the % IA/g of anti-PD-1 nine days after i.p. treatment is diminished in multiple tissues including kidneys, spleen, liver and bones of tumor-bearing mice compared to healthy counterparts, possibly due to the systemic immunosuppressive effect of intracranial tumors that result in a dearth of circulating lymphocyte populations that are able to bind the anti-PD-120. Despite the systemic immunosuppressive response in glioma-bearing mice, injecting hydrogel and anti-PD-1 admixture in the lymph nodes allowed focused delivery of anti-PD-1 to the site of anti-tumor priming (lymph nodes) and anti-tumor activity (brain), allowing amelioration of immune response. 1.5.2 Lymph nodes exposed to sustained anti-PD-1 show increased immunogenic activity compared to those exposed to systemic anti-PD-1

To assess the immunogenic potential of anti-PD-1 checkpoint blockade in a GBM model, mice were administered with either i.p. (systemic) therapeutic anti-PD-1 or hydrogel-loaded therapeutic anti-PD-1. The former was given over three time points as described in previous studies, Kim et al., 2017, while mice treated with hydrogels were given a one-time injection in one of the following locations: intracranial at the site of the tumor, the left inguinal region or the left anterior cervical region. Due to spatial limitations with the murine intracranial compartment, a max dose of 200 μs in 6 μL of hydrogel was given. All other mice, however, were treated with a total of 600 μg of anti-PD-1 in 50 μL of hydrogel solution.

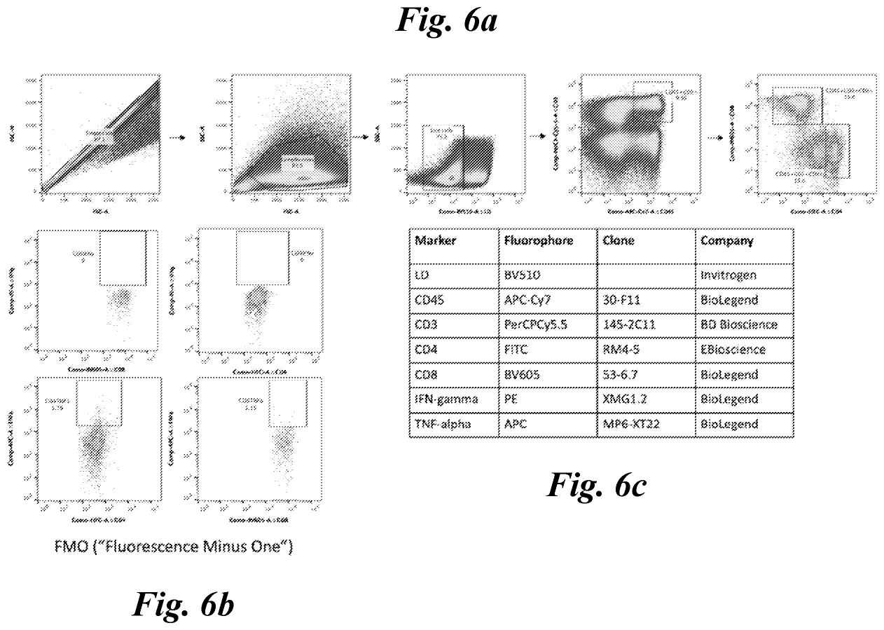

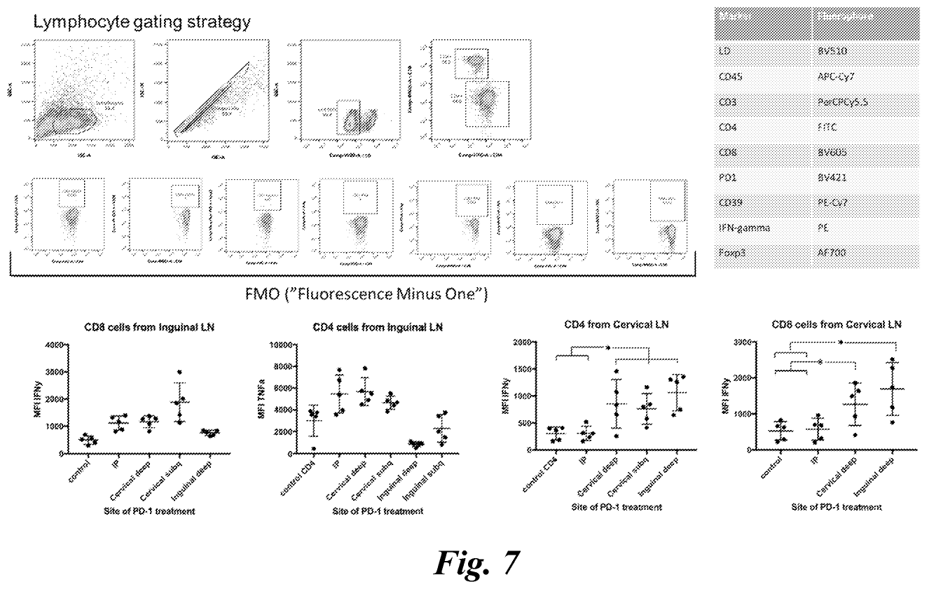

Gating strategies with markers for assessing CD4+ and CD8+ T cell activation were used to assess for the immunogenic potential of the hydrogel platform (FIG. 6a and FIG. 7). Fluorescence minus one (FMO) for IFN-γ and TNF-α, immune activation markers that are upregulated in activated immune cells, such as CD4+ and CD8+ T lymphocytes (FIG. 6b and FIG. 7), were utilized to indicate proportion of activated cell populations. Overall, there was greater IFN-γ and TNF-α activity from CD4+ and CD8+ T cells in the brains of mice treated with hydrogels in the inguinal and cervical regions. Notably, mice that were given hydrogels loaded with anti-PD-1 in the intracranial space showed comparable immunogenic changes to control mice without treatment. Due to issues with limited volume in the intracranial space, however, these changes might reflect the lower dose of anti-PD-1 (200 μg vs 600 μg) (FIG. 3b-FIG. 3d).

The overall trend of hydrogels demonstrating increased activation of CD4+ and CD8+ T lymphocytes is significant, with both IFN-γ and TNF-α showing increased representation in CD4+ and CD8+ populations in inguinal and cervical hydrogel models. In regard to IFN-γ specifically, there was an increase in percentage of CD4+ IFN-γ cells in the brain in mice treated with hydrogels in the deep cervical lymph node space compared to mice treated with systemically with i.p. anti-PD-1 (P=0.0007) (FIG. 3a-FIG. 3b). Similarly, mice treated with hydrogels in the inguinal area also demonstrated increased expression of CD4+ IFN-γ cells compared to mice treated with i.p. anti-PD-1 (P=0.0430). There was no significant difference in CD4+ IFN-γ expression in mice treated with hydrogels, however, in either the inguinal or deep cervical lymph node regions (FIG. 3a-FIG. 3b). The expression of CD8+ IFN-γ-producing cells was highest in tumor-bearing mice treated with hydrogels loaded with anti-PD-1 in the deep cervical region compared to i.p. injected mice (P=0.0468) and inguinal hydrogel mice (P=0.0229). Interestingly, there was no significant difference in CD8+ IFN-γ-producing cell populations between mice treated via i.p. or by hydrogel in the inguinal region (FIG. 3a-FIG. 3b). While TNF-α showed a similar overall trend for inguinal and cervical hydrogel models having increased activation of their lymphocyte compartments compared to i.p. treated mice, there was actually a trend toward more activation in the inguinal lymph node hydrogel system rather than with direct cervical lymph nodes. When examining CD4+ and CD8+ TNF-α binding cells, there was no significant difference between either hydrogel group (P=0.3134 and P=0.6507, respectively) (FIG. 3c, FIG. 3d). There was a statistically significant difference, however, between CD4+ and CD8+ TNF-α binding cells in the brain between i.p. treated and inguinal lymph node hydrogel mice (P=0.0316 and P=0.0044, respectively) (FIG. 3c-FIG. 3d).

1.5.3 Survival Efficacy is Improved with Localized Sustained Delivery to Lymph Nodes

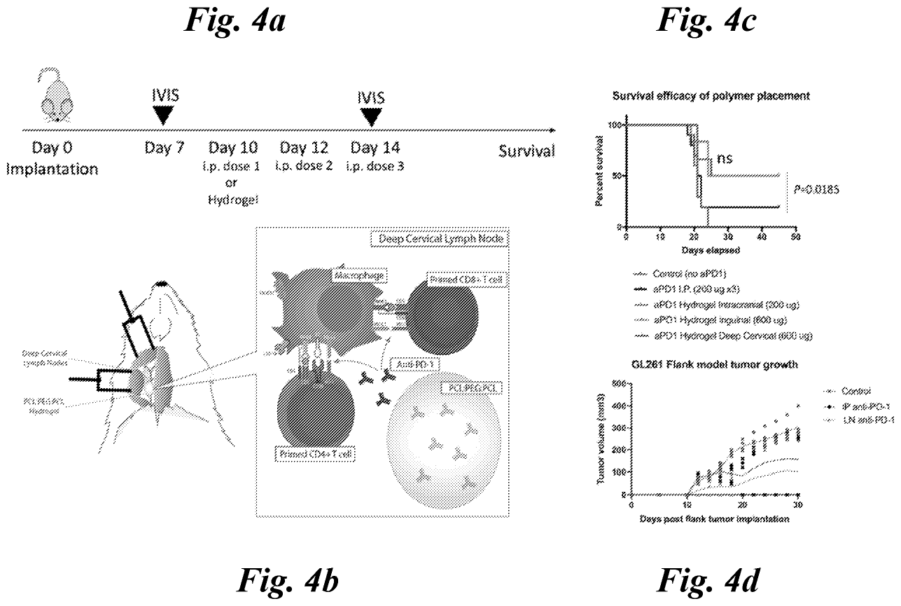

To assess for the therapeutic efficacy of sustained and localized delivery of anti-PD-1 to lymph nodes, mice were implanted with GL261-Luc2 and selected for similar tumor burden via IVIS® before being randomly assorted to one of five arms: control (no treatment), i.p. systemic anti-PD-1 treatment (three 200 μg doses), anti-PD-1 loaded into a hydrogel in the intracranial space at the site of the tumor (one-time 200 μg dose), anti-PD-1 loaded into a hydrogel at the inguinal region (one-time 600 μg dose), and anti-PD-1 loaded into a hydrogel at the deep cervical lymph node region (one-time 600 μg dose) (FIG. 4a-FIG. 4b).

Overall, mice with hydrogels carrying anti-PD-1 at the inguinal and deep cervical lymph node regions exhibited improved overall survival compared to i.p. injected mice (P=0.0185) (FIG. 4c). There was no significant difference in therapeutic efficacy in mice treated with hydrogels in the inguinal or cervical region. Survival efficacy was similar between control and intracranial hydrogel mice, with the intracranial compartment limiting the dosage and thereby diminishing the comparative insight of this finding. Moreover, when examining the effect of hydrogel placement on local tumor growth in flank models involving GL261, there was a clear advantage in overall decreased rate of tumor growth in mice with hydrogel placement near the tumor. Intriguingly, despite establishing radiographic tumor burden through IVIS® on post-implantation day 7, three of the five mice that were implanted with hydrogel carrying anti-PD-1 did not develop measurable tumor burden (FIG. 4d).

### 1.5.4 Polymer Based Delivery to Lymph Nodes Results in Increased IFN-γ in Co-Culture

To further examine the immunophenotypic changes that result from the long-term local presence of anti-PD-1 at the site of lymph nodes, isolated CD3+ T lymphocytes were harvested from the inguinal and deep cervical lymph nodes of mice treated with hydrogels at the inguinal and cervical site, respectively. Inguinal and cervical lymph nodes of mice without any treatment and mice with systemic anti-PD-1 therapy (i.p. injections) were used as comparative controls. These immune cells were co-cultured with dendritic cells and tumor cell lysate, with ELISA used to assess for production of IFN-γ.

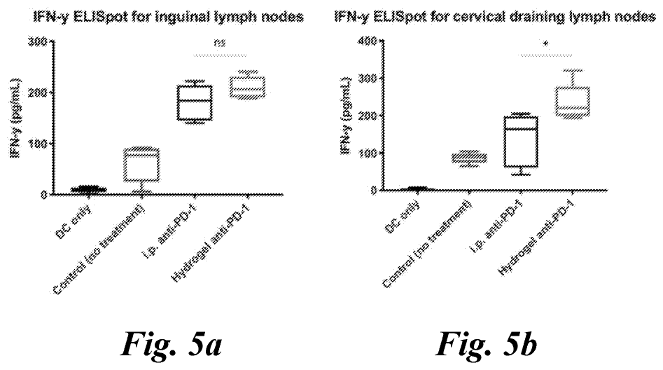

In mice implanted with hydrogels at the inguinal region, there was no statistically significant difference between the expression of IFN-γ in CD3+ T lymphocytes between i.p. and hydrogel-treated arms (FIG. 5a). There was a significant increase, however, in IFN-γ expression in CD3+ lymphocytes from the deep cervical draining lymph nodes in mice that had hydrogels in the anterior cervical space compared to those that had i.p. therapy (P=0.0379) (FIG. 5b).

### 1.6 Discussion

The relatively recent advent of checkpoint blockade as an exciting therapeutic avenue has resulted in successes for multiple cancers, though there are important headways yet to be made in optimizing treatments. Currently, the main method of delivery for checkpoint blockade agents, such as anti-PD-1, involves systemic delivery, which requires multiple infusions that may result in systemic toxicities. The presently disclosed subject matter demonstrates proof of principle that sustained and localized delivery of immunotherapeutic agents to lymph nodes is a viable therapeutic option in preclinical models, with resultant reversal of immunosuppression as demonstrated by increased IFN-γ and TNF-α activity in T lymphocytes, as well as increased therapeutic efficacy compared to the current model of multiple systemic infusions (FIG. 4). Moreover, the presently disclosed subject matter demonstrates in a preclinical model that there also is less delivery of anti-PD-1 to other organs with hydrogel-based therapy, with the majority of anti-PD-1 being released to the local lymph node and the site of tumor (FIG. 1 and FIG. 2). Additionally, there was unexpected insight into how anti-PD-1 traverses the blood-brain barrier (BBB) and localizes to the brain specifically in mice with tumors, otherwise exhibiting a marginal presence in the healthy brain (FIG. 1 and FIG. 2). It is unclear at this time, however, if the antibody is being bound to immune cells and trafficking to the brain, if the free antibody itself traverses the compromised blood-brain barrier, or if a combination of both events occurs.

Recently, there has been much interest in targeting GBM locally in the intracranial compartment with both chemotherapy and immunotherapy. The presently disclosed subject matter, however, offers an alternative target that prescribes local therapy to lymph nodes, in this case the deep cervical and inguinal lymph nodes. Interestingly, while there was a greater trend toward reversing immunosuppression with treatment of the deep cervical lymph nodes when looking at IFN-γ expression alone, the therapeutic efficacy of targeting either the inguinal or deep cervical lymph nodes was similar in a murine model. This observation might be, in part, accounted for by other activating cytokines, such as TNF-α, that showed trends toward increased activation of lymphocytes in inguinal lymph nodes compared to cervical lymph nodes. In either case, however, placement of anti-PD-1 releasing hydrogels in inguinal and cervical lymph nodes demonstrates increased activation of CD4+ and CD8+ populations compared to mice injected systemically with anti-PD-1. While there are many more avenues to explore regarding local lymph node delivery, the presently disclosed data demonstrate proof of principle of targeting lymph nodes with sustained delivery as a viable target for reactivating immune cells.

Finally, the present observations regarding accumulation of anti-PD-1 in the brain selectively over other non-lymphoid tissues in the context of tumors could be explained by prior studies in immune cell homeostasis and trafficking. In previous studies, T cell activation and expansion against tumor-derived antigens in the draining lymph nodes was observed over many days following adoptive transfer of tumor-specific T cells in murine models of glioma (including on day 5). Kim et al., 2017; Jackson et al., 2016; Garzon-Muvdi et al., 2018. Because these anti-tumor T cells are concentrated in the lymph nodes and exposed to local anti-PD-1 during activation, it is thought that upon infiltrating the tumor they allow accumulation of anti-PD-1 in the brain gradually. On the other hand, it is suspected that i.p. treated mice do not have adequate binding of anti-PD-1 as they would be binding to more diffuse circulating tumor-reactive T cells; this is further challenged by the relatively short half-life of circulating anti-PD-1 (22.3 hours). Zalba et al., 2020. As mentioned before, there are likely differences in immune cell populations and activating cytokines between inguinal and cervical lymph nodes that were not able to be fully explored in the scope of this study.

Several studies have supported the concept of targeted therapies of lymph nodes. For instance, tumor-specific CD8+ T cells have long since been understood as undergoing activation in the tumor-draining lymph nodes, with the potential to differentiate into anti-tumor effector phenotypes occurring at these robust immune sites. Prokhnevska et al., 2020.

Moreover, it has already been well established that tumor-specific T cell responses are initiated in lymph nodes where antigen presentation is occurring. Rotman et al., 2019. In both mouse models and humans, CD103+ (mouse) and CD141+ (human) migratory dendritic cells were found to carry tumor antigens to lymph nodes and cross-present them to CD8+ T lymphocytes. Roberts et al., 2016; Salmon et al., 2016. Moreover, this is all in light of the unforgiving nature of the physical tumor microenvironment of GBM—with its hypoxic and necrotic features, as well as tumorigenic stromal neighbors—that presents an ongoing challenge with local immunotherapy at the site of the tumor. Jackson et al., 2019. As such, the slightly less immunosuppressive environment of lymph nodes offers a much more enviable target for reversing immunosuppression. Topalian et al., 2020.

Previous studies have suggested that anti-tumor T cell responses can be promoted more robustly in lymph nodes distal from brain tumors, but only in the context of local injection of tumor-vaccines, which overcomes the hurdle of antigen drainage to distal lymph nodes. Ohlfest et al., 2013. In the present study, it may be possible that inguinal-injected hydrogel acts as a depot to deliver anti-PD-1 to lymphocytes enroute to the site of anti-tumor priming in more proximal tumor draining lymph nodes; this might also explain the lower proportion of lymph node T cell activation in the inguinal lymph nodes (FIG. 5). There also are studies, however, that suggest that inguinal lymph nodes may communicate with spinal cerebrospinal fluid (CSF) and also act as surrogate tumor-draining lymph nodes to the CNS compartment, which may in turn that may have antigen presentation Ma et al., 2019. Investigating the nature of CD4 and CD8 activation following anti-PD-1 at distal and proximal lymph nodes is warranted to further understand their respective roles in the rejuvenation of an anti-tumor response.

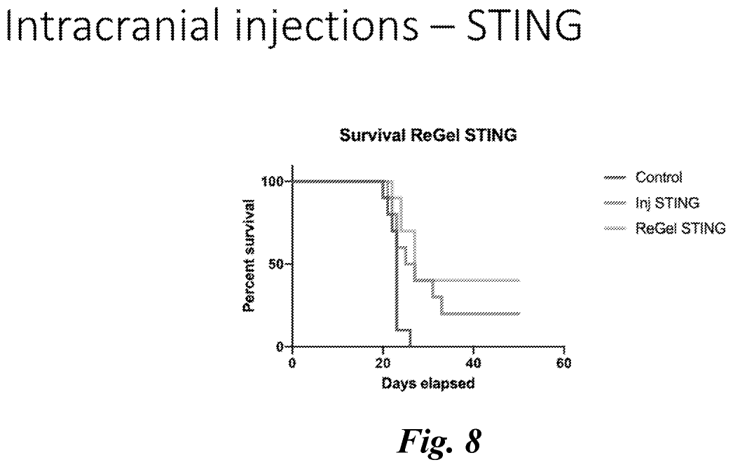

Finally, while anti-PD-1 was used as a proof of principle for delivering sustained and localized immunotherapy, the potential for using a depot form for delivering other immunomodulating therapies, such as stimulator of interferon genes (STING) agonists (see, for example, FIG. 8, to target myeloid cells at the level of the lymph node also holds potential. Limitations of this study included lack of side effect profiles to determine whether there was mitigation of irAEs with localized therapy to lymph nodes of interest, though this also was a reflection of the limits of the current mouse model. Additionally, the dosage discrepancy between intracranial (limited by the enclosed compartment of the mouse cranium) and lymph node anti-PD-1 hydrogel delivery makes it difficult for meaningful comparison. Furthermore, to test the robustness of local and sustained immunotherapeutic delivery to lymph nodes, additional agents that target different immune compartments within lymph nodes would have offered more detailed information.

Finally, while the presently disclosed study showed proof of principle for targeting lymph nodes using sustained release of immunotherapy as a viable treatment avenue, more in-depth analysis of the different cell populations and their interactions involving immunosuppressive and activating cytokines at the level of different lymph nodes should be explored in future studies. Future directions would likely include combinatorial immunotherapy strategies that target the tumor draining lymph nodes for neoadjuvant access to the immune compartment with accompanying surgical resection, chemotherapy, and radiation.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
McKenzie M, Betts D, Suh A, Bui K, Kim LD, Cho H. Hydrogel-Based Drug Delivery Systems for Poorly Water-Soluble Drugs. Molecules. 2015; 20(11):20397-20408.
Vellimana, A. K., Recinos, V. R., Hwang, L., Fowers, K. D., Li, K. W., Zhang, Y., Okonma, S., Eberhart, C. G., Brem, H., and Tyler, B. M., Combination of paclitaxel thermal gel depot with temozolomide and radiotherapy significantly prolongs survival in an experimental rodent glioma model., J. Neurooncol. 2013, 111(3):229-236.
Larrañeta E, Stewart S, Ervine M, Al-Kasasbeh R, Donnelly R F. Hydrogels for Hydrophobic Drug Delivery. Classification, Synthesis and Applications. J Funct Biomater. 2018; 9(1):13.
Goldberg M V, Maris C H, Hipkiss E L, Flies A S, Zhen L, Tuder R M, Grosso J F, Harris T J, Getnet D, Whartenby K A, et al. Role of PD-1 and its ligand, B7-H1, in early fate decisions of CD8 T cells. Blood. 2007; 110(1):186-9.
Tsushima F, Yao S, Shin T, Flies A, Flies S, Xu H, Tamada K, Pardoll DM, Chen L. Interaction between B7-H1 and PD-1 determines initiation and reversal of T-cell anergy. Blood. 2007; 110(1):180-185.
Topalian S L, Taube J M, Pardoll D M. Neoadjuvant checkpoint blockade for cancer immunotherapy. Science. 2020; 367(80):6477.
Blackburn S D, Crawford A, Shin H, Polley A, Freeman G J, Wherry E J. Tissue-specific differences in PD-1 and PD-L1 expression during chronic viral infection: implications for CD8 T-cell exhaustion. J Virol. 2010; 84(4):2078-2089.
Khan B T. Pembrolizumab for patients with advanced melanoma. Lancet Oncol. 2015; 16(6):e264.
Reck M, Rodriguez-Abreu D, Robinson A G, Hui R, Csöszi T, Fülöp A, Gottfried M, Peled N, Tafreshi A, Cuffe S, et al. Pembrolizumab versus chemotherapy for PD-L1-positive non-small-cell lung cancer. N Engl J Med. 2016; 375(19):1823-1833.
Larkin J, Chiarion-Sileni V, Gonzalez R, Grob -J-J, Rutkowski P, Lao C D, Cowey C L, Schadendorf D, Wagstaff J, Dummer R, et al. Five-year survival with combined nivolumab and ipilimumab in advanced melanoma. N Engl J Med. 2019; 381(16):1535-1546.
Jackson C M, Choi J, Lim M. Mechanisms of immunotherapy resistance: lessons from glioblastoma. Nat Immunol. 2019; 20(9):1100-1109.
Woroniecka K, Chongsathidkiet P, Rhodin K, Kemeny H, Dechant C, Farber S H, Elsamadicy A A, Cui X, Koyama S, Jackson C, et al. T-cell exhaustion signatures vary with tumor type and are severe in glioblastoma. Clin Cancer Res. 2018; 24(17):4175-4186.
Almutairi A R, McBride A, Slack M, Erstad BL, Abraham I. Potential immune-related adverse events associated with monotherapy and combination therapy of ipilimumab, nivolumab, and pembrolizumab for advanced melanoma: a systematic review and meta-analysis. Front Oncol. 2020:10.
Kim J E, Patel M A, Mangraviti A, Kim E S, Theodros D, Velarde E, Liu A, Sankey E W, Tam A, Xu H, et al. Combination therapy with anti-PD-1, anti-TIM-3, and focal radiation results in regression of murine gliomas. Clin Cancer Res. 2017; 23(1):124-136.
Rotman J, Koster B D, Jordanova E S, Heeren A M, de Gruijl T D. Unlocking the therapeutic potential of primary tumor-draining lymph nodes. Cancer Immunol Immunother. 2019; 68(10):1681-1688.
Roberts E W, Broz M L, Binnewies M, Headley M B, Nelson A E, Wolf D M, Kaisho T, Bogunovic D, Bhardwaj N, Krummel M F, et al. Critical role for CD103+/CD141+ dendritic cells bearing CCR7 for tumor antigen trafficking and priming of T cell immunity in melanoma. Cancer Cell. 2016; 30(2):324-336.
Salmon H, Idoyaga J, Rahman A, Leboeuf M, Remark R, Jordan S, Casanova-Acebes M, Khudoynazarova M, Agudo J, Tung N, et al. Expansion and activation of CD103+dendritic cell progenitors at the tumor site enhances tumor responses to therapeutic PD-L1 and BRAF inhibition. Immunity. 2016; 44(4):924-938.
Fransen M F, Schoonderwoerd M, Knopf P, Camps M G M, Hawinkels L J A C, Kneilling M, van Hall T, Ossendorp F. Tumor-draining lymph nodes are pivotal in PD-1/PD-L1 checkpoint therapy. JCI Insight. 2018; 3(23):23.
Strauss L, Mahmoud M A A, Weaver J D, Tij aro-Ovalle N M, Christofides A, Wang Q, Pal R, Yuan M, Asara J, Patsoukis N, et al. Targeted deletion of PD-1 in myeloid cells induces antitumor immunity. Sci Immunol. 2020; 5(43):43.
Zeng J, See A P, Phallen J, Jackson C M, Belcaid Z, Ruzevick J, Durham N, Meyer C, Harris T J, Albesiano E, et al. Anti-PD-1 blockade and stereotactic radiation produce long-term survival in mice with intracranial gliomas. Int J Radiat Oncol. 2013; 86(2):343-349.
Hirano F, Kaneko K, Tamura H, et al. Blockade of B7-H1 and PD-1 by monoclonal antibodies potentiates cancer therapeutic immunity. Cancer Res. 2005 Feb. 1; 65(3):1089-96.
Brechbiel M W, Gansow O A. Synthesis of C-functionalized trans-cyclohexyldiethylenetriaminepenta-acetic acids for labelling of monoclonal antibodies with the bismuth-212 α-particle emitter. J Chem Soc Perkin Trans. 1992; 1(9):1173-1178.
Chongsathidkiet P, Jackson C, Koyama S, Loebel F, Cui X, Farber S H, Woroniecka K, Elsamadicy A A, Dechant C A, Kemeny H R, et al. Sequestration of T cells in bone marrow in the setting of glioblastoma and other intracranial tumors. Nat Med. 2018; 24(9):1459-1468.
Jackson C M, Kochel C M, Nirschl C J, Durham N M, Ruzevick J, Alme A, Francica B J, Elias J, Daniels A, Dubensky T W, et al. Systemic tolerance mediated by melanoma brain tumors is reversible by radiotherapy and vaccination. Clin Cancer Res. 2016; 22(5):1161-1172.
Garzon-Muvdi T, Theodros D, Luksik A S, Maxwell R, Kim E, Jackson C M, Belcaid Z, Ganguly S, Tyler B, Brem H, et al. Dendritic cell activation enhances anti-PD-1 mediated immunotherapy against glioblastoma. Oncotarget. 2018; 9(29):20681-20697.
Zalba S, Contreras-Sandoval A M, Martisova E, Debets R, Smerdou C, Garrido M J. Quantification of pharmacokinetic profiles of pd-l/pd-l1 antibodies by validated elisas. Pharmaceutics. 2020; 12(6):1-16.
Prokhnevska N, Cardenas M, Jansen C, et al. Tumor-specific CD8 T cell activation in draining lymph nodes supports the anti-tumor CD8 T cell response. J Immunol. 2020; 204(1Supplement).
Ohlfest J R, Andersen B M, Litterman A J, Xia J, Pennell C A, Swier L E, Salazar A M, Olin M R. Vaccine injection site matters: qualitative and quantitative defects in CD8 T cells primed as a function of proximity to the tumor in a murine glioma model. J Immunol. 2013; 190(2):613-620.
Ma Q, Schlegel F, Bachmann S B, et al. Lymphatic outflow of cerebrospinal fluid is reduced in glioma. Sci Rep. 2019; 9(1):1-10.

Although the foregoing subject matter has been described in some detail by way of illustration and example for purposes of clarity of understanding, it will be understood by those skilled in the art that certain changes and modifications can be practiced within the scope of the appended claims.

1. A method for treating a glioblastoma (GBM), the method comprising administering a composition comprising a hydrogel and an anti-programmed cell death protein 1 (PD-1) antibody to one or more draining lymph nodes (DLNs) of a subject in need of treatment thereof.

2. The method of claim 1, wherein the one or more DLNs comprise a cervical lymph node or an inguinal lymph node.

3. The method of claim 1, wherein the hydrogel comprises an ABA block tripolymer.

4. The method of claim 3, wherein the B block of the ABA block tripolymer comprises poly(ethylene glycol) (PEG).

5. The method of claim 3, wherein the A block of the ABA block tripolymer comprises one or more hydrophobic polymers.

6. The method of claim 5, wherein the one or more hydrophobic polymers are selected from poly(ε-caprolactone) (PCL), poly(D,L-lactide-co-glycolic acid) (PLGA), poly (D,L-lactic acid) (PLA), poly(p-phenylene oxide) (PPO), polyhydroxybutyrate (PHB), and combinations thereof.

7. The method of claim 1, wherein the hydrogel comprises a poly(ε-caprolactone)-b-poly(ethylene glycol)-b-poly(ε-caprolactone) (PCL:PEG:PCL) triblock polymer or a poly(lactide-co-glycolide)-b-poly(ethylene glycol)-b-poly(lactide-co-glycolide) (PLGA-PEG-PLGA) triblock polymer.

8. The method of claim 1, wherein the hydrogel comprises a thermosensitive hydrogel.

9. The method of claim 8, wherein the hydrogel further comprises one or more pH-sensitive moieties.

10. The method of claim 1, wherein the anti-PD-1 antibody is selected from cemiplimab, nivolumab, pembrolizumab, avelumab, atezolizumab, and combinations thereof.

11. The method of claim 1, further comprising administering the hydrogel composition in combination with one or more therapies for treating a GBM.

12. The method of claim 11, wherein the one or more therapies for treating a GBM are selected from surgical resection, surgical re-resection, radiation therapy, chemotherapy, vaccine therapy, oncolytic viral therapy, steroid therapy, laser interstitial thermal therapy (LITT), tumor treating fields (TTF) therapy, laser ablation, one or more additional immunotherapies, CSF-1R inhibition, TGF-beta inhibition, IDO-1 inhibition, stromal vascular fraction (SVF) stem cell therapy, stimulator of type-I interferon (IFN) genes) (STING) agonist, and combinations thereof.

13. The method of claim 12, wherein the radiation therapy comprises one or more of X-ray radiation, gamma ray radiation, and proton beam radiation therapy.

14. The method of claim 12, wherein the radiation therapy comprises one or more of intensity-modulated radiation therapy (IMRT), tomotherapy, stereotactic radiosurgery, hypofractionated stereotactic radiosurgery, stereotactic radiosurgery with valproic acid, and pencil beam proton therapy.

15. The method of claim 12, wherein the chemotherapy comprises an alkylating agent.

16. The method of claim 15, wherein the alkylating agent is selected from temozolomide, lomustine, carmustine, procarbazine, vincristine, and combinations thereof.

17. The method of claim 12, wherein the steroid therapy comprises or more of dexamethasone, prednisone, and combinations thereof.

18. The method of claim 12, wherein the one or more additional immunotherapies are selected from one or more additional checkpoint inhibitors, one or more vascular endothelial growth factor (VEGF) antagonists, one or more cytotoxic T-lymphocyte-associated protein 4 (CTLA-4) inhibitors, one or more vascular endothelial growth factor receptor (VEGFR) inhibitors, CAR-T cell therapy, and combinations thereof.

19. The method of claim 18, wherein the one or more checkpoint inhibitors is selected from anti-PD-L1, anti-LAG-3, anti-CD137, anti-CD-27, intratumoral IDO1 inhibitor, IDO1 inhibitor, and combinations thereof.

20. The method of claim 18, wherein the one or more VEGF antagonists comprises bevacizumab.

21. The method of claim 18, wherein the one or more CTLA-4 inhibitors are selected from ipilimumab, tremelimumab, and combinations thereof.

22. The method of claim 18, wherein the one or more vascular endothelial growth factor receptor (VEGFR) inhibitors comprises cediranib.

23. The method of claim 1, wherein the GBM is selected from O-6-methylguanine-DNA methyltransferase gene (MGMT)-methylated GBM and GBM having unmethylated/indeterminate MGMT promoter status.

24. The method of claim 1, wherein the method comprises one or more of a reversal of immunosuppression, an increase in levels of IFN-γ and TFN-α in T lymphocytes, an increased activation of CD4+ and CD8+ T lymphocytes, and combinations thereof, relative to a level of immunosuppression, an increase in a level of IFN-γ and TFN-α in T lymphocytes, and/or a level of activation of CD4+ and CD8+ T lymphocytes in the subject before administration of the composition comprising a hydrogel and an anti-programmed cell death protein 1 (PD-1) antibody to one or more draining lymph nodes (DLNs) of the subject.
